(19) United States
(12) Patent Application Publication
(10) Pub. No.: US 2003/0228366 A1
(43) Pub. Date: Dec. 11, 2003

(54) Reconstitutable compositions of biodegradable block copolymers

(76) Inventors: Chung Shih, Sandy, UT (US); Gaylen M. Zentner, Salt Lake City, UT (US); Ai-Zhi Piao, Salt Lake City, UT (US)

Correspondence Address: M. Wayne Western, THORPE, NORTH & WESTERN, L.L.P., P.O. Box 1219, Sandy, UT, 84091-1219, US

(21) Appl. No.: 10/167,768

(22) Filed: Jun. 11, 2002

(51) Int. Cl. A61K 9/14

(52) U.S. Cl. 424/486

(57) ABSTRACT

A composition having enhanced reconstitution properties and method of use is disclosed. The composition comprises one or more biodegradable block copolymer drug carriers; and a reconstitution enhancing and enabling agent comprising polyethylene glycol (PEG), a PEG derivative or a mixture of PEG and a PEG derivative; and wherein the biodegradable block copolymer drug carrier is soluble in an aqueous solution and in the liquid reconstitution enhancing and enabling agent.

### FIELD OF THE INVENTION

The present invention relates to reconstitutable compositions comprising a water soluble, low molecular weight polyethylene glycol (PEG), PEG derivatives, or mixtures of PEG and PEG derivatives and their use for facilitating the reconstitution of biodegradable block copolymeric drug carriers in an hydrophilic environment. Particularly, this invention relates to flowable compositions comprising a water soluble polyethylene glycol (PEG), PEG derivatives, or a mixture of a PEG and a PEG derivative, and biodegradable ABA, BAB and AB type block copolymers that are based on biodegradable polyester or poly(ortho ester) A blocks and polyethylene glycol (PEG) B blocks. The composition can be rapidly reconstituted in an aqueous vehicle to afford useful forms that may be either homogeneous solutions or uniform colloidal systems.

### BACKGROUND OF THE INVENTION

Biodegradable polymers have been used as surgical sutures, wound dressings, and as drug delivery systems. Among them, polylactide (PLA), polyglycolide (PGA) and their copolymers (PLGA) have attracted the most attention. One example of a biodegradable polymeric drug delivery system is a system wherein a drug is contained in a biodegradable polymer matrix that is surgically implanted, which is a big disadvantage. In the form of injectable drug delivery systems, polymeric microspheres and nanospheres are known in the art. Commercially available drug delivery formulations based on PLGA microspheres include Lupron Depot® and Nutropin Depot®. Microsphere and nanosphere systems have disadvantages in that they require special and complex preparation methods. Unfortunately, manufacturing microsphere and nanosphere dosage forms requires use of toxic or dangerous solvents (e.g., methylene chloride, ethyl acetate) and elaborate procedures (e.g., double emulsions, or cryogenic spraying techniques). The batch size is usually small and the cost is high. In addition, since PLGA biodegradable polymers used can only be dissolved in organic solvents their preparation requires the use of such solvents which are foreign and harmful to the human body, and cannot be completely removed during manufacture by any known method. Furthermore, some drugs such as peptides and proteins may lose their pharmacological activity after contact with organic solvents.

An improvement to the aforementioned drug delivery systems is an in situ formed depot based on PLGA as disclosed in U.S. Pat. No. 5,599,552. In that system, PLGA is dissolved in water-soluble organic solvent(s), such as N-methyl-2-pyrrolidone, and the drug is either suspended or dissolved in this polymeric solution. The solution can be injected subcutaneously to form an in situ depot to trap the drug in the polymer that precipitates as the organic solvent diffuses away. However, the drawback is the requirement for an organic solvent that is used to dissolve the biodegradable PLGA polymer. Organic solvents, such as N-methyl-2-pyrrolidone, are foreign to the human body and can cause unwanted side effects both acutely and chronically.

U.S. Pat. No. 5,543,158 discloses nanoparticles or microparticles formed from a water-insoluble block copolymer consisting essentially of poly(alkylene glycol) and poly(lactic acid). The molecular weight of the block copolymer is high and the copolymer is insoluble in water. In the nanoparticle or microparticle, the biodegradable moieties of the copolymer are in the core of the nanoparticle or microparticle and the poly(alkylene glycol) moieties are on the surface of the nanoparticle or microparticle in an amount effective enough to decrease uptake of the nanoparticle or microparticle by the reticuloendothelial system. Nanoparticles are prepared by dissolving the block copolymer and drug in an organic solvent, forming an o/w emulsion by sonication or stirring, and collecting the nanoparticles containing the drug following precipitation.

Currently there are few synthetic or natural polymeric materials that can be used for the controlled delivery of drugs, including peptide and protein drugs, because of strict regulatory compliance requirements such as biocompatibility, low toxicity, having a clearly defined degradation pathway, and safety of the polymers and degradation products. The most widely investigated and advanced biodegradable polymers in regard to available toxicological and clinical data are the aliphatic poly(α-hydroxy acids), such as poly(D-, L-, or D, L-lactic acid) (PLA), poly(glycolic acid) (PGA) and their copolymers (PLGA). These polymers are commercially available and are presently used as bioresorbable sutures and in biodegradable microsphere drug delivery systems. FDA-approved microsphere systems for controlled release of leuprolide acetate (Lupron Depot™) and human growth hormone (Nutropin Depot™) are based on PLGA copolymers. Based on this history of use, PLGA copolymers have been the materials of choice in the initial design of parenteral controlled release drug delivery systems using a biodegradable carrier.

Even though there has been some limited success, biodegradable block copolymers that are based on biodegradable polyester or poly(ortho ester) and polyethylene glycol (PEG) blocks, when used as drug carriers, present problems that are associated with their physicochemical properties and attendant methods of fabrication. For example, biodegradable block copolymers are, by design, not stable in aqueous environments although shelf-lives of several years can be achieved when they are stored frozen. However, elimination of cold storage requirements would be advantageous in most instances. It is also desirable to gain further advantages related to rapid dissolution of neat block copolymers into aqueous vehicles at normal room temperature conditions. Rapid dissolution of the block copolymers permits reconstitution at time-of-use to occur, which in turn permits room temperature storage of neat block copolymers. Known water soluble block copolymers are slow to dissolve in water, often requiring several hours for complete dissolution to occur. Compositions that show accelerated dissolution kinetics are desired.

Some drugs, such as proteins, are stable in aqueous solutions for only short periods. To compensate for this short-term stability, these drugs are commonly formulated as dry cakes and powders that can be stored under water-free conditions for much longer periods. Immediately prior to administration the dry cake or powder is reconstituted with an aqueous vehicle. Thus the situation is frequently encountered where it is desirable to have both the drug and the block copolymer drug delivery system formulated in reconstitutable forms. To be facile, it is critical that reconstitution, i.e., dissolution of the block copolymers and drug be completed in a short period. There has been no previous disclosure of compositions comprising (a) a water soluble, low molecular weight PEG, PEG derivatives (i.e., lactide and/or glycolide derivatized PEG), or a mixture of a PEG and a PEG derivative, and (b) a biodegradable ABA, BAB or AB type block copolymer that is based on biodegradable polyester or poly(ortho ester) A blocks and PEG B blocks. The compositions can be rapidly reconstituted in an aqueous vehicle to afford a homogeneous true solution or uniform colloidal system. Accordingly, the present invention represents improved drug delivery compositions that minimize or are free of the problems mentioned above.

### SUMMARY OF THE INVENTION

The present invention provides compositions that can be rapidly reconstituted in an aqueous vehicle to afford a homogeneous solution or uniform colloidal system, and methods of use thereof for preparing a pharmaceutically effective formulation for delivery of drugs.

The present invention also provides a method for effective reconstitution of a drug delivery composition in an aqueous vehicle to afford a homogeneous solution or uniform colloidal system and a method for effectively administering such a composition to warm blooded animals. The administration can be done by any functional means such as parenteral, ocular, inhalation, transdermal, vaginal, buccal, transmucosal, transurethral, rectal, nasal, oral, peroral, pulmonary, topical or aural and any other means of administration that may be compatible with the present invention.

The composition of the present invention comprises: 1) one or more biodegradable block copolymer drug carriers comprising A-B, A-B-A or B-A-B block copolymers having a total molecular weight of 2000 to 8000 Daltons, wherein the A block is a biodegradable polyester or poly(ortho ester) and the B block is polyethylene glycol (PEG), and the weight percentage of the A block is between 50.1% to 83%; and 2) a reconstitution enhancing and enabling agent comprising a liquid polyethylene glycol (PEG), a PEG derivative, or a mixtures of PEG and a PEG derivative, said PEG or PEG derivative having a weight averaged molecular weight of 150 to 1100 Daltons; wherein the biodegradable drug carrier is soluble in an aqueous solution and in the PEG and/or PEG derivatives.

The biodegradable block copolymer drug carriers suitable of the present invention can form homogeneous, free-flowing solutions or uniform colloidal systems in water when present from 1% up to 40% by weight. Reconstitution is the process of mixing an agent to be reconstituted with a solvent, which in the case of pharmaceuticals is usually aqueous. After reconstitution the mixture may exist in the final physical state as either a true solution or a uniform colloidal or suspension system. The time course for achieving the final physical state of the mixture should be rapid and facile. The present invention relates to compositions that enable rapid reconstitution of block copolymeric drug carriers to the final physical state as either a true solution or a uniform colloidal system. By “rapid” is meant that the reconstitution process occurs within a short period of time, typically between 0.01 minutes to 120 minutes, preferably within 0.01 minutes to 60 minutes, and most preferably within 0.01 minutes to 30 minutes.

The reconstitution enhancing and enabling agents of the present invention have low-viscosity, and are water-soluble liquids that have a good affinity with both water and the block copolymeric drug carriers, and yet permit the block copolymeric drug carriers to function in the desired manner at the administration site in a human or other warm-blooded animal. Particularly, low molecular weight PEG, or lactide and/or glycolide derivatized low molecular weight PEG, or mixtures thereof, are good candidates for the reconstitution enhancing and enabling agents of the present invention.

Examples of suitable biodegradable water soluble drug carriers includes biodegradable ABA- or BAB-type triblock copolymers, or AB-type diblock copolymers based on biodegradable polyester or poly(ortho ester) A-blocks and hydrophilic B polymer block(s) consisting of polyethylene glycol (PEG). The biodegradable polyester are synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, malic acid, and copolymers thereof.

Polyethylene glycol (PEG) is also sometimes referred to as poly(ethylene oxide) (PEO) or poly(oxyethylene) when incorporated into a block copolymer, and the terms can be used interchangeably for the purposes of this invention.

In the case where the A-block(s) are PLA/PLGA polyester, the lactate content is between about 20 to 100 mole percent, preferably between about 50 to 100 mole percent. The glycolate content is between about 0 and 80 mole percent, preferably between about 0 to 50 mole percent. Or, stated differently, when the A-block is PLGA the glycolate content is between about 1 and 80 mole percent and preferably between about 1 and 50 mole percent and the lactate content is between 20 and 99 mole percent and preferably between 50 and 99 mole percent.

The compositions of the present invention are effective in reconstituting water soluble block copolymeric drug delivery compositions in aqueous vehicles to afford homogeneous solutions or uniform colloidal systems thus facilitating administration of a uniform and accurate dose that may then, in many cases, enhance the therapeutic effect of the drug. Homogeneous solutions and uniform colloidal systems drug delivery compositions includes all free flowing forms of the compositions comprising biodegradable block copolymer drug carriers and reconstitution enhancing and enabling agents of the present invention, water, drug(s), and any additives or excipients as necessary to prepare formulations that are pharmaceutically and therapeutically useful. The drug may be present as either a true solution or in a colloidal state such as emulsion or a suspension. All forms can act to facilitate administration of the drug and enhance the therapeutic effect. Such therapeutic effects may be optimized by controlling the copolymer molecular weights, compositions, and the relative ratios of the hydrophilic and hydrophobic blocks, ratios of drug to copolymer, ratios of copolymer to PEG and/or PEG derivatives, and both drug and copolymer concentrations in the final administered dosage form. Additional advantages of this invention will become apparent from the following detailed description of the various embodiments.

### DETAILED DESCRIPTION OF THE INVENTION

This invention is not limited to the particular configurations, process steps, and materials disclosed herein, as such configurations, process steps, and materials may vary somewhat. It is also to be understood that the terminology employed herein is used for the purpose of describing particular embodiments only, and is not intended to be limiting since the scope of the present invention will be limited only by the appended claims and equivalents thereof.

In this specification and the appended claims, the singular forms “a,” “an,” and “the” include plural references unless the context clearly dictates otherwise. Thus, for example, reference to a composition for delivering “a drug” includes reference to one, two, or more drugs. In describing and claiming the present invention, the following terminology will be used in accordance with the definitions set out below.

“Effective amount” means an amount of a drug, biologically active agent or pharmacologically active agent that provides the desired local or systemic effect.

“Reconstitution” refers to mixing of biodegradable block copolymer drug carriers and the reconstitution enhancing and enabling agents with an aqueous solvent system to create a homogenous solution or uniform colloidal system. This is in addition to the more traditional definition of reconstitution where drug and excipients are mixed with a solvent, usually aqueous, immediately before administration. When the present invention is fully utilized, the reconstituted block copolymer system can be used to reconstitute a drug. The combination of block copolymer(s) and reconstitution enhancing and enabling agents can be easily reconstituted with an aqueous solvent system as described above.

“Copolymer solution”, when used in reference to a biodegradable block copolymer contained in such a solution, shall mean an aqueous composition having such biodegradable block copolymer drug carrier and the reconstitution enhancing and enabling PEG and/or PEG derivatives either dissolved to form a homogeneous solution or uniform colloidal system. As such, copolymer solution includes all free flowing forms of the composition of the present invention and water.

“Reconstitutable compositions” and/or “compositions having enhanced reconstitution properties” shall mean compositions comprising biodegradable block copolymer drug carrier, and the reconstitution enhancing and enabling agents comprising low molecular weight PEG, PEG derivatives, or mixtures of PEG and PEG derivatives.

“Reconstitutable drug formulations”, “reconstitutable drug delivery compositions”, “drug delivery compositions having enhanced reconstitution properties” and the like, shall mean the combination of drug, the block copolymer drug carrier, and the reconstitution enhancing and enabling agents comprising low molecular weight PEG, PEG derivatives, or mixtures of PEG and PEG derivatives. They shall include all combinations of the drug with the block copolymer and reconstitution enhancing and enabling agents, for example block copolymer solutions that are mixed with the drug to form drug solutions, as well as mixtures of undissolved block copolymers with the drug, i.e. block reconstitutable copolymeric drug delivery compositions, that are subsequently reconstituted into an aqueous environment to form a drug containing block copolymer solution.

“Enhanced reconstitution properties” refers to properties that enable rapid reconstitution of block copolymeric drug carriers to the final physical state as either a true solution or a uniform colloidal system. The reconstitution process occurs within a short period of time, typically between 0.01 minutes to 120 minutes, preferably within 0.01 minutes to 60 minutes, and most preferably within 0.01 minutes to 30 minutes.

“Aqueous solution”, “solution”, “homogeneous solution” and the like, shall include water without additives or aqueous solutions containing additives or excipients such as pH buffers, components for tonicity adjustment, antioxidants, preservatives, drug stabilizers, etc., as commonly used in the preparation of pharmaceutical formulations.

“Drug solution”, “solubilized drug”, “dissolved drug” and all other terms that refer to the drug in a solution or dissolved state includes the drug being present as either a homogeneous solution, micellar solution, or in a colloidal state such as emulsion or a suspension. Thus, solubilized drugs and drug solutions include all free flowing forms of the compositions comprising the reconstitution enhancing and enabling agent, biodegradable block copolymer drug carriers, water and drug(s). All forms can act to facilitate administration of the drug and enhance the therapeutic effect.

“Administration” is the means by which drug formulations are presented to humans and other warm-blooded animals in effective amounts, and includes all routes for dosing or administering drugs, whether self-administered or administered by medical practitioners.

“Parenteral” shall mean administration by means other than through the digestive tract such as by intramuscular, intraperitoneal, intra-abdominal, subcutaneous, intrathecal, intrapleural, intravenous and intraarterial means.

“Reverse thermal gelation” is the phenomenon whereby a solution of a block copolymer spontaneously increases in viscosity, and in many instances transforms into a semisolid gel, as the temperature of the polymer solution is increased above the gelation temperature of the polymer solution. For the purpose of the invention, the term gel includes both the semisolid gel state and the high viscosity state that exists above the gelation temperature. When cooled below the gelation temperature the gel spontaneously reverses to reform the lower viscosity polymer solution. This cycling between the solution and the gel may be repeated indefinitely because the sol/gel transition does not involve any change in the chemical composition of the polymer solution. All interactions to create the gel are physical in nature and do not involve the formation or breaking of covalent bonds.

“Depot” means a liquid drug delivery system that forms a gel upon the temperature being raised to or above the gelation temperature following administration to a warm-blooded animal.

“Gel” means the semi-solid phase that spontaneously occurs as the temperature the liquid block copolymeric composition is raised to or above the gelation temperature.

“Gel mixture” or “mixture of triblock copolymers” refers to a reverse thermal gelation system comprising two or more ABA or BAB triblock copolymer components. The mixture can be made either by simply mixing two or more individually synthesized triblock copolymer components, or by synthesizing two or more types of copolymer systems in one synthesizing vessel. The mixture prepared by the above two processes may be combined with water to form a polymer solution that may have the same or different gelation properties and gel qualities.

“Biodegradable” means that the block copolymer or oligomer can chemically break down or degrade within the body to form nontoxic components. The rate of degradation can be the same or different from the rate of drug release.

“Drug” shall mean any organic or inorganic compound or substance having biological or pharmacological activity that can be adapted or used for a therapeutic purpose.

“Peptide,” “polypeptide,” “oligopeptide” and “protein” shall be used interchangeably when referring to peptide or protein drugs and shall not be limited as to any particular molecular weight, peptide sequence or length, field of bioactivity or therapeutic use unless specifically stated.

“PLGA” shall mean a copolymer or copolymer radicals derived from the condensation copolymerization of lactic acid and glycolic acid, or, by the ring opening copolymerization of lactide and glycolide. The terms lactic acid and lactate are used interchangeably; glycolic acid and glycolate are also used interchangeably.

“PLA” shall mean a polymer derived from the condensation of lactic acid or by the ring opening polymerization of lactide.

“PGA” shall mean a polymer derived from the condensation of glycolic acid or by the ring opening polymerization of glycolide.

“Biodegradable polyester or poly(ortho ester)s” refers to any biodegradable polyester or poly(ortho ester)s, the polyesters are preferably synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acid, malic acid, and copolymers thereof.

The present invention is based on the discovery of a reconstitution enhancing and enabling agent for biodegradable block copolymer drug carriers which can in minutes efficiently accelerate the dissolution of the drug carriers into an aqueous medium to create an injectable copolymer solution or drug solution. The “reconstitution enhancing and enabling agent” of the present invention comprises a PEG or PEG derivatives having a weight averaged molecular weight of 150 to 1100, or a mixture of a PEG and a PEG derivative. The PEG derivative refers to a PEG having a molecular weight of 150 to 1100 and is derived from a member selected from the group consisting of D,L-lactide, D-lactide, L-lactide, D,L-lactic acid, D-lactic acid, L-lactic acid, glycolide, glycolic acid and copolymers thereof. The PEG derivative can also be a member represented by R¹—CO—O-(PEG)-CO—R² or R¹—O(PEG)-R² wherein R¹ and R² are independently members selected from the group consisting of H and C₁ to C₁₀ alkyl and the PEG has a molecular weight of 150 to 1100. The biodegradable block copolymer drug carriers of the present is soluble both in an aqueous solution and in the PEG and/or PEG derivatives. Examples of these biodegradable block copolymer drug carriers are disclosed in U.S. Pat. No. 6,201,072 and pending U.S. patent application Ser. Nos. 09/559,799; 09/971,074 and 09/971,082, hereby fully incorporated by reference.

In one embodiment, the biodegradable drug carrier comprises ABA-type or BAB-type triblock copolymers, AB-type diblock copolymers or mixtures thereof, where the A-blocks are relatively hydrophobic and comprises a biodegradable polyester or poly(ortho ester), and the B-blocks are relatively hydrophilic and comprises polyethylene glycol (PEG), said copolymer having a hydrophobic content of between 50.1 to 83% by weight and hydrophilic content of between 17 to 49.9% by weight, and an overall block copolymer molecular weight of between 2000 and 8000. The drug carriers exhibit water solubility at temperatures below normal mammalian body temperatures and undergoes reversible thermal gelation to then exist as a gel at temperatures equal to physiological mammalian body temperatures.

In another embodiment, the biodegradable drug carrier is an ABA-type, BAB-type, or AB-type block copolymer, or mixtures thereof, where the A-blocks are relatively hydrophobic and comprises a biodegradable polyester or poly(ortho ester), and the B-blocks are relatively hydrophilic and comprises polyethylene glycol (PEG), said block copolymer having a hydrophobic content of between 50.1 to 65% by weight and a hydrophilic content of between 35 to 49.9% by weight, and an overall block copolymer weight-averaged molecular weight of between 2400 and 4999. The drug carriers are water soluble and capable of enhancing the solubility of drugs, hydrophobic drugs in particular, in water, to form a drug solution.

In still another embodiment, the polymeric drug carrier comprises biodegradable polyester or poly(ortho ester) oligomers, and particularly PLA/PLGA oligomers having a weight averaged molecular weight of between 400 and 10,000, mixed with biodegradable ABA-type or BAB-type triblock copolymers, or AB-type diblock copolymers having a weight averaged molecular weight of between 2400 and 4999. The block copolymers have 50.1 to 65% by weight of the hydrophobic A block(s) comprising biodegradable polyester or poly(ortho ester)s and 35 to 49.9% by weight of the hydrophilic B block(s) consisting of polyethylene glycol (PEG).

It is also within the scope of the invention to include any drug carrying compositions that can be reconstituted with an aqueous vehicle to create an aqueous formulations but the time needed for reconstitution is undesirably long. It is an unexpected discovery that the composition of the present invention can significantly decrease the reconstitution time and increase the water solubility and the stability of the drug in the formulations. It is also surprising that inclusion an additional reconstitution enabling and enhancing agent of the present invention further enhances the dissolution rate of the formulations.

The compositions of the present invention have good gelation, solubilization performance, or combined properties. In general, the reconstitution enhancing and enabling agents of the present invention are water-soluble, low molecular weight liquid polyethylene glycols(PEG), PEG derivatives, or mixtures of PEG and PEG derivatives. In one particular embodiment, lactide and/or glycolide derivatized low molecular weight PEG or mixtures PEG and PEG derivatives are suitable reconstitution enhancing agents for biodegradable block copolymer drug carriers. In other embodiments, the reconstitution enhancing and enabling agent is lactide and/or glycolide derivatized PEG having a molecular weight between 150 to 1100, wherein the PEG molecular weight is 150 to 600; the PLGA:PEG weight ratio is 0.01 to 0.75. When the PEG derivative is derivatized by both glycolide and lactide, the glycolide content is 0.01 to 99.99%, preferably 1 to 75%; and the lactide content is 0.01 to 99.99%, preferably 25 to 99%. The PEG derivatives of the present invention also include PLA wherein the lactide content is 0.01 to 100% and PGA wherein the glycolide content is 0.01 to 100%.

The reconstitutable compositions as disclosed herein, have compositional make-ups within the indicated ranges that render the composition rapidly reconstitutable. For purposes of disclosing molecular weight parameters, all reported molecular weight values are based on measurements by ¹H-NMR or GPC (gel permeation chromatography) analytical techniques. The reported weight averaged molecular weights and number averaged molecular weights were determined by GPC and ¹H-NMR, respectively. The reported lactide/glycolide ratios were calculated from ¹H-NMR data. GPC analysis was performed on a Styragel HR-3 column, or equivalent, calibrated with PEG standards using RI detection and chloroform as the eluent, or on a combination of Phenogel, mixed bed, and 500 Å columns calibrated with PEG standards using RI detection and tetrahydrofuran as the eluent for the ABA and BAB triblock copolymers.

ABA-type and BAB-type triblock copolymers, and AB-type diblock copolymers may be synthesized by ring opening polymerization, or condensation polymerization. Additionally, the B-blocks may, in certain instances, be coupled to the A-blocks by ester or urethane links and the like. Condensation polymerization and ring opening polymerization procedures may be utilized as may the coupling of a monofunctional hydrophilic B block to either end of a difunctional hydrophobic A block in the presence of coupling agents such as isocyanates. Furthermore, coupling reactions may follow activation of functional groups with activating agents, such as carbonyl diimidazole, succinic anhydride, N-hydroxy succinimide, p-nitrophenyl chloroformate and the like.

The hydrophilic B-block is formed from PEG of an appropriate molecular weight. PEG was chosen as the hydrophilic B-block because of its unique biocompatibility, nontoxic properties, hydrophilicity, solubilization properties, and rapid clearance from a patient's body. The hydrophobic A-blocks are utilized because of their biodegradable, biocompatible, and solubilization properties. The in vitro and in vivo degradation of hydrophobic, biodegradable polyester or poly(ortho ester) A-blocks are well understood and the degradation products are readily metabolized and/or eliminated from the patient's body.

Thus, water soluble biodegradable block copolymers are prepared wherein the hydrophilic B-block(s) make up about 17 to 49.9% by weight of the copolymer and the hydrophobic A-block or blocks make up about 50.1 to 83% by weight of the copolymer. The weight ratio of the biodegradable block copolymer drug carrier and the reconstitution enhancing and enabling agent(s), particularly water soluble low molecular weight PEG, PEG derivatives, or mixtures of PEG and PEG derivatives, is between 2:1 and 1:99.

The composition of the present invention can be quickly reconstituted in water or an aqueous solution and form a polymer solution comprising the composition of the present invention in water or the aqueous solution at a weight ratio between 2:1 and 1:1000.

Drugs that may be delivered by the reconstitutable drug delivery compositions of the present invention can be any bioactive agent, but particular advantage is achieved with bioactive agents having limited solubility or dispersibility in an aqueous or hydrophilic environment, or any bioactive agent that requires enhanced solubility or dispersibility. Without limiting the scope of the present invention, suitable drugs include those drugs presented in current edition of Goodman and Gilman's “The Pharmacological Basis of Therapeutics” or the current edition of The Merck Index. Both volumes list drugs suitable for numerous types of therapeutic applications, including drugs in the following categories:drugs acting at synaptic and neuroeffector junctional sites, drugs acting on the central nervous system, drugs that influence inflammatory responses, drugs that affect the composition of body fluids, drugs affecting renal function and electrolyte metabolism, cardiovascular drugs, drugs affecting gastrointestinal function, drugs affecting uterine motility, chemotherapeutic agents for parasitic infections, chemotherapeutic agents for microbial diseases, antineoplastic agents, immunosuppressive agents, drugs affecting the blood and blood-forming organs, hormones and hormone antagonists, dermatological agents, heavy metal antagonists, vitamins and nutrients, vaccines, oligonucleotides and gene therapies.

Incorporating one or more drugs mentioned in the above categories with the compositions of the present invention to form drug delivery compositions which can be easily reconstituted to form an aqueous solution or uniform colloidal system can be achieved by simply adding the drug to an aqueous solutions of the compositions of the present invention, or by mixing the drug with the compositions of the present invention and thereafter adding water or an aqueous solution to form a solution or uniform colloidal system.

Mixtures of the compositions of the present invention with peptide/protein drugs, and/or other types of drugs, may be prepared as reconstitutable drug delivery formulations that may be easily reconstituted in the form of a solution or dispersion. This aqueous formulation is then administered parenterally, topically, transdermally, transmucosally, inhaled, or inserted into a cavity such as by ocular, vaginal, transurethral, rectal, nasal, oral, peroral, buccal, pulmonary or aural administration to a patient. Many of the aqueously solubilized drug formulations prepared by implementing the present invention may be diluted in an i.v. bag or by other means, and administered to a patient for an extended period, without precipitation of the drug. Due to the biocompatibility of the materials and the free flowing nature of the system at physiological temperatures, this system will cause minimal toxicity and minimal mechanical irritation to the surrounding tissue.

A distinct advantage to the compositions of this invention lies in the ability of the water soluble, reconstitution enhancing and enabling agents to reduce the viscosity of the water soluble biodegradable block copolymer drug carriers into a form that is quickly reconstitutable in water or an aqueous solution to form a polymer solution for drug delivery. In one possible configuration, a dosage form comprised of a solution of the block copolymer drug carrier and a reconstitution enhancing and enabling agent that contains dissolved drug is administered to the body. Before administration, the reconstitutable drug containing composition may be freeze-dried for long-term storage, and the lyophilized biodegradable polymeric drug composition may be quickly reconstituted to its original solution by using water or other aqueous solutions.

The only limitation as to how much drug can be dissolved or dispersed in the reconstitutable drug delivery composition of the present invention is one of functionality, namely, the drug:copolymer ratio may be increased until the properties of the mixture are adversely affected to an unacceptable degree, or until the properties of the system are adversely affected to such a degree as to make administration of the system unacceptably difficult. Generally speaking, it is anticipated that in most instances where dissolution is desired, the drug will be present at between about 10⁻⁶ to about 100 percent by weight of the combined weight the block copolymer drug carrier and the reconstitution enhancing and enabling agents, with ranges of between about 0.001% to 25% by weight being the most common. For example, having the drug present at 100% by weight combined weight the block copolymer drug carrier and the reconstitution enhancing and enabling agents means that the drug and combined weight the block copolymer drug carrier and the reconstitution enhancing and enabling agents are present in equal amounts (i.e., equal weights). Generally speaking, it is anticipated that in most instances where dispersion is desired, the upper drug:polymer ratio could substantially exceed the range noted above for dissolution. These ranges of drug loading are illustrative and will include most drugs that may be utilized in the present invention. However, such ranges are not limiting to the invention should drug loadings outside this range be functional and effective.

The present invention thus provides reconstitutable compositions comprising biodegradable block copolymer drug carriers and reconstitution enhancing and enabling agents that can be rapidly reconstituted in an aqueous vehicle to afford useful forms that may be either homogeneous true solutions or uniform colloidal systems. The drug solution formed with the reconstitutable drug delivery compositions of the present invention has desirable physical stability, therapeutic efficacy, and toxicology. The reconstitution enhancing and enabling agents of the present invention can be used for any water soluble drug carriers, particularly for biodegradable di- or triblock copolymers that have reverse gelation properties and/or polymers that can enhance the solubility of drugs, especially hydrophobic drugs.

The following are examples that illustrate preferred embodiments of the invention but are intended as being representative only.

### EXAMPLE 1

This example illustrates the synthesis of a reconstitution enhancing and enabling agent in the present invention.

PEG-300 (107.6 g) was placed in a 250-mL round bottom flask and dried under vacuum (0.2 torr, 90° C.) for 3 hours. D,L-Lactide (33.4 g) and glycolide (9.0 g) was added and the head-space was replaced by dried nitrogen. The mixture was brought to 135° C. and the reaction was initiated by adding stannous octoate (20 mg) via a dry syringe. The reaction mixture was allowed to stir under dry nitrogen at 155° C. for four additional hours. Residual monomers were removed under vacuum (0.2 torr, 90° C., 2 hr). The product was a clear free-flowing liquid.

### EXAMPLE 2

This example illustrates the synthesis of the ABA-type triblock copolymer PLGA-PEG-PLGA by ring opening copolymerization.

PEG 1450 (476.2 g) was dried under vacuum (1 mmHg) at 130° C. for 5 hours. D, L-Lactide (412.9 g) and glycolide (110.9 g) were added to the flask and heated to 145° C. to afford a homogenous solution. Polymerization was initiated by the addition of 250 mg stannous octoate to the reaction mixture. After maintaining the reaction for five hours at 145° C., the reaction was stopped and the flask was cooled to room temperature. Unreacted lactide and glycolide were removed by vacuum distillation. The resulting PLGA-PEG-PLGA copolymer had a weight averaged molecular weight (Mw) of 3855 as measured by GPC. The GPC was performed on two Phenogel columns (300×7.8), 500 Å, and a mixed bed connected in series. Mobile phase was tetrahydrofuran and peaks were detected by a differential refractory index detector. The chromatograms were calibrated against PEG standards. This triblock copolymer possessed the property of enhancing the aqueous solubility of drugs and, in particular, hydrophobic drugs. This triblock copolymer did not form a gel at or below 37° C.

### EXAMPLE 3

This example illustrates the synthesis of the ABA-type triblock copolymer PLGA-PEG-PLGA by ring opening copolymerization.

PEG 1000 NF (65.3 g) and PEG 1450 NF (261 g) was dried under vacuum (1 mmHg) at 130° C. for 5 hours. D, L-Lactide (531.12 g) and glycolide (142.6 g) were added to the flask and heated to 145° C. to afford a homogenous solution. Polymerization was initiated by the addition of 250 mg stannous octoate to the reaction mixture. After maintaining the reaction for five hours at 145° C., the reaction was stopped and the flask was cooled to room temperature. Unreacted lactide and glycolide were removed by vacuum distillation. The resulting PLGA-PEG-PLGA copolymer had a weight averaged molecular weight (Mw) of 4255 as measured by GPC. A 23% by weight aqueous solution of this triblock copolymer had a gel temperature at 32.8° C.

### EXAMPLE 4

The reconstitution enhancing and enabling properties of PEG derivatives are illustrated in this example.

The PEG derivatives prepared in Example 1 (1.5 g) were added to 1 gram of PLGA-PEG-PLGA triblock copolymer prepared in Example 3. The two components were intimately mixed into a homogeneous mixture. To the mixture, water for injection (5 g) was added. The mixture was subjected to wrist shaking at 100 strokes/minute. The solution was visually inspected every 30 seconds for homogeneity. The time required for mixing was reported as the reconstitution time. The mixture described above took 1 minute to reconstitute. The resulting aqueous solution had a gelation temperature of 33.1° C.

### EXAMPLE 5

Following the method described in Example 4, using the triblock copolymer to reconstitution enhancing and enabling agent ratio described in Table 1, it was noted that the reconstitution time decreased with increasing weight ratio of reconstitution enhancing and enabling agent in the mixture. The same triblock copolymer without reconstitution-enhancing and enabling agent took long than 5 hours to reconstitute (Entry 1). It was also noted that the reconstitution time was reduced when quantity of water was increased.

TABLE 1
Reconstitution time as a function of weight ratio between
Component I (triblock copolymer described in Example 3) and
Component II (PEG derivative described in Example 1) and water.

| | Component | Component | Water for | Tgel | Reconstitution |
|---|---|---|---|---|---|
| Entry | I (g) | II (g) | injection (g) | (° C.) | Time (min) |
| 1 | 1 | 0 | 6.5 | 31.5 | 480 |
| 2 | 1 | 1 | 5.5 | 32.0 | 15 |
| 3 | 1 | 1.25 | 5.25 | 32.2 | 5 |
| 4 | 1 | 1.5 | 5 | 33.1 | 1 |
| 5 | 1 | 1.75 | 4.75 | 35.5 | 1 |
| 6 | 1 | 1.5 | 2.5 | 28.0 | 5 |
| 7 | 1 | 1.5 | 6.25 | 34.2 | 1 |
| 8 | 1 | 1.5 | 10 | 35.5 | .5 |

### EXAMPLE 6

This example illustrates the synthesis of an ABA-type PLGA-PEG-PLGA triblock copolymer by condensation copolymerization.

Into a three-necked flask, equipped with a nitrogen inlet, thermometer, and distillation head for removal of water, was placed D,L-lactic acid (360 g) and glycolic acid (96.7 g). The reaction mixture was heated under nitrogen at 160° C., with stirring, for three days. The resulting PLGA copolymer had a weight averaged molecular weight (Mw) of 8800.

The PLGA copolymer (165 g) was mixed with PEG 1450 NF (150 g) and was heated in a flask at 160° C. under a nitrogen atmosphere. After 7 days, the reaction was stopped and the flask was cooled to room temperature. The residue was a high viscosity liquid. The resulting PLGA-PEG-PLGA block copolymer had a weight averaged molecular weight (Mw) of 3910 determined by the GPC method described in Example 2. The triblock copolymer possessed the property of enhancing the aqueous solubility of drugs and, in particular, hydrophobic drugs.

Reconstitution using PEG 200, PEG 300 or agent prepared in Example 1, by the method described in Example 4, at triblock copolymer to reconstitution enhancing and enabling agent ratio of 1:1.5, and water to the mixture ratio of 2:1, took 1 minute.

### EXAMPLE 7

This example illustrates the synthesis of an ABA-type PLGA-PEG-PLGA triblock copolymer by condensation copolymerization.

Into a three-necked flask, equipped with a nitrogen inlet, thermometer, and distillation head for removal of water, was placed D,L-lactic acid (360 g) and glycolic acid (96.7 g). The reaction mixture was heated under nitrogen at 160° C., with stirring, for three days. The resulting PLGA copolymer had a weight averaged molecular weight (Mw) of 8800. PLGA (505 g), PEG 1000 NF (49.0 g) and PEG 1450 NF (195.8 g) were heated in a flask at 160° C. under a nitrogen atmosphere for 7 days. The reaction was stopped and the polymeric residue was thick and amber in color. The residue (ca. 100 g) was purified by dissolving in 400 mL of water at ambient and precipitating at 70° C. The aqueous layer was discarded and this cycle was repeated one more time. Excess water in the residue was removed by lyophilization, the resulting polymer had a weight-averaged molecular weight of 4015. Aqueous solution (23%) of the polymer had a gelation temperature of 33.8° C. Reconstitution of this triblock copolymer was measured by the method described in Example 4. Thus, 1 gram of the triblock copolymer was intimately mixed with reconstitution enhancing and enabling agent described in Example 1 (1.5 g). Reconstitution of this mixture in 5 mL of water took 1 minute and the resulting solution had a gelation temperature of 32.5° C.

### EXAMPLE 8

AB diblock copolymers were synthesized by placing 35.7 g of PEG-Me (Mw: 2000) in a 250 mL 3-neck round bottom reaction flask. Water was removed by heating in an oil bath (155° C.) under vacuum (0.5 torr) for 3 hours. The reaction flask was then raised out of the oil bath and the vacuum was released.

D,L-Lactide (32.0 g) was weighed and added to the reaction flask. The headspace was replaced with dry nitrogen by repeated evacuation and flushing with dry nitrogen 5 times.

The flask was then lowered and immersed in a 155° C. oil bath. Once the content was melted and the internal temperature reached 150° C., 2 drops (200 ppm) of stannous 2-ethylhexanoate was added to initiate the polymerization. The reaction mixture was stirred using an overhead stirrer for 8 hours at a rate of 100-200 rpm. The temperature was then reduced to 140° C., and the residual monomer was removed under reduced pressure (<1 torr) over 1 hour. The residue was a translucent, off-white solid having a molecular weight of 4550. The polymer possessed the property of enhancing the solubility of drugs and, in particular, hydrophobic drugs. Reconstitution time was measured using the agent prepared in Example 1 at ratios described in Example 4. The reconstitution time was 1 minute and the resulting solution did not form a gel at or below 37° C.

### EXAMPLE 9

Methoxy-PEG (MW 550; 48.6 g) was transferred into a 250 mL 3-neck round bottom reaction flask. The oil bath was heated to 100° C. The molten PEG-Me was stirred under vacuum for 5 hours to remove water. The reaction flask was then raised outside of the oil bath and the vacuum was released. D,L-Lactide (97.68 g) and glycolide (26.47 g) were weighed and added the reaction flask. The headspace was replaced with dry nitrogen. The flask was then immersed into a 155° C. oil bath. Once the D,L-lactide was melted and the temperature inside the reaction flask reached 150° C., 2 drops (200 ppm) of stannous 2-ethylhexanoate was added to the reaction flask. The reaction was stirred continuously for 8 hours at a rate of 100-150 rpm.

The oil bath temperature was reduced to 140° C. and the reaction flask was attached to vacuum (<1 torr) for an hour to remove residual monomer. The diblock copolymer had honey-like consistency with molecular weight of 2010. The residue (145 g) was added to 1,6-diisocynatohexane (6.06 g) via an oven dried syringe and the reaction mixture was allowed to stir at 140° C. for 2 additional hours. The residue was purified by precipitation twice from water at 70° C. Water was removed by lyophilization and the residual BAB triblock copolymer had a molecular weight of 4250. An aqueous solution (23%) of the polymer had a gelation temperature of 31.2° C. Reconstitution time was measured using the agent prepared in Example 1 at ratios described in Example 4. The reconstitution time was 1 minute and the resulting solution had a gelation temperature of 32.8° C.

Although diblock copolymers are coupled via urethane linkages in the current example, ester links, or a combination of ester and urethane linkages are adequate for coupling two diblock polymeric molecules.

### EXAMPLE 10

Following the same procedures outlined in Example 1, fifteen reconstitution enhancing agents based on the type of poly(ethylene glycol) and stoichiometry described in Table 2, were synthesized. These reconstitution enhancing and enabling agents were mixed individually with the triblock copolymer prepared in Example 3 at a ratio of triblock copolymer to reconstitution enhancing and enabling agent of 1:1.5 and water to the mixture ratio of 2:1, to afford a free-flowing mixture. Reconstitution times of these mixtures, using the method described in Example 4, and the gelation temperatures were also reported in Table 2.

TABLE 2
Stoichiometry of additional reconstitution enhancing and enabling agents prepared
from the PEG, D,L-lactide and glycolide, the reconstitution time required when
mixing PLG-PEG-PLG triblock copolymer prepared in Example 3 with these
reconstitution enhancing and enabling agents, and gelation temperature.
The ratio of triblock copolymer to reconstitution enhancing and enabling
agent ratio was 1:1.5, respectively. The mixtures (2.5 g each) were
reconstituted into 5 mL of water.

| | | PEG weight | Glycolide | D,L-Lactide | T_{gel} | Reconstitution |
|---|---|---|---|---|---|---|
| Entry | PEG | (gram) | (gram) | (gram) | (° C.) | Time (min) |
| 1 | PEG200NF | 30.0 | 7.62 | 28.38 | — | 1.0 |
| 2 | PEG200NF | 33.33 | 5.64 | 21.02 | — | 1.0 |
| 3 | PEG200NF | 50.0 | 6.35 | 23.65 | — | 1.0 |
| 4 | PEG200NF | 107.14 | 9.07 | 35.79 | 35.9 | 1.0 |
| 5 | PEG200NF | 50.0 | 2.12 | 7.88 | 30.7 | 1.5 |
| 6 | PEG300NF | 57.14 | 4.84 | 18.02 | 32.1 | 1.0 |
| 7 | PEG400NF | 86.15 | 24.67 | 9.19 | 32.1 | 1.0 |
| 8 | PEG600NF | 50.0 | 4.23 | 15.75 | 35.1* | 1.0 |
| 9 | Triethylene | 50.0 | 4.23 | 15.77 | 32.2 | 1.0 |
| | glycol | | | | | |
| 10 | Tetraethylene | 111.11 | 8.23 | 30.66 | 30.5 | 1.5 |
| | glycol | | | | | |
| 11 | PEG300NF | 50.25 | 19.75 | — | 31.1 | 1.0 |
| 12 | PEG300NF | 86.15 | 24.67 | 9.19 | 35.3 | 1.0 |
| 13 | PEG300NF | 86.15 | 15.1 | 18.75 | 37.4 | 1.0 |
| 14 | PEG300NF | 143.58 | 11.94 | 44.48 | 38.8 | 1.0 |
| 15 | PEG300NF | 100.5 | — | 39.5 | 40.6 | 1.0 |

*Viscous solution at ambient temperature
Without these agents, the reconstitution time of the said polymer was longer than 5 hours.

### EXAMPLE 11

This example illustrates the use of the reconstitution enhancing agent of the present invention in reconstituting a paclitaxel formulation wherein the drug carrier is the PLGA-PEG-PLGA triblock copolymer described in Example 2.

The PEG derivatives prepared in Example 1 (1.5 g) were added to 1 gram of PLGA-PEG-PLGA triblock copolymer described in Example 2. Also added to the mixture was 50 mg of paclitaxel. The mixture was intimately mixed into a homogeneous mixture at ca. 40° C. After cooling to ambient temperature, water for injection (5 g) was added to the mixture. The mixture was subjected to wrist shaking at approximately 100 strokes/min. The solution was visually inspected every 30 seconds for homogeneity. Complete dissolution was confirmed by aspirating and pushing it through a ½′ 26-gauge needle using a 1-mL insulin syringe. The time required for reconstitution was 1 minute.

### COMPARATIVE EXAMPLE OF EXAMPLE 11

Paclitaxel (50 mg) was dissolved in the PLGA-PEG-PLGA triblock copolymer (1 g) described in Example 2 without using the reconstitution-enhancing and enabling agent of the present invention. Water for injection (6.5 g) was added to the mixture. The mixture was subjected to wrist shaking at approximately 100 strokes/min for 5 minutes and the content was not dissolved. A magnetic stirring bar was placed and the mixture was stirred at ca. 250 rpm at ambient for least 5 hours to afford a homogeneous solution. The solution did not form a gel at or below 37° C.

### EXAMPLE 12

The PEG derivatives prepared in Example 1 (1.5 g) were intimately mixed with 1 gram of PLGA-PEG-PLGA triblock copolymer described in Example 2 and 0.08 g of poly(D,L-lactate-co-glycolate) (MW 1200) into a homogeneous mixture. Paclitaxel (75 mg) was dissolved into the mixture with gentle stirring at ca. 45° C. After equilibrated to ambient temperature, water for injection (5 g) was added and the mixture was subjected to wrist shaking at approximately 100 strokes/min. The solution was visually inspected every 30 seconds for homogeneity. Complete dissolution was confirmed by aspirating through a ½′ 26-gauge needle using a 1-mL insulin syringe. The time required for complete dissolution was 1 minute. The solution did not form a gel at or below 37° C.

### EXAMPLE 13

The PEG derivatives prepared in Example 1 (1.5 g) were added to 1 gram of Me-PEG-PLGA diblock copolymer described in Example 8. Also added to the mixture was 50 mg of paclitaxel. The mixture was intimately mixed into a homogeneous mixture. Water for injection (5 gram) was added to the mixture. The mixture was subjected to wrist shaking at approximately 100 strokes/min. The solution was visually inspected every 30 seconds for homogeneity. Complete dissolution was confirmed by aspirating and pushing it through a ½′ 26-gauge needle using a 1-mL insulin syringe. The time required for reconstitution was 1 minute. The solution did not form a gel at or below 37° C.

### COMPARATIVE EXAMPLE OF EXAMPLE 13

Paclitaxel (50 mg) was dissolved in Me-PEG-PLGA diblock copolymer (1 gram) described in Example 8 without the reconstitution-enhancing agent of the present invention. Water for injection (6.5 gram) was added to the mixture. The mixture was subjected to wrist shaking at 100 strokes/min for 5 minutes and the content was not dissolved. A magnetic stirring bar was placed and the mixture was stirred at ca. 250 rpm at ambient for least 5 hours to afford a homogeneous solution. The solution did not form a gel at or below 37° C.

### EXAMPLE 14

Paclitaxel (1.04 g) was added to the PEG derivatives described in Example 1 (40.0 g). The mixture was warmed to 40° C. to ensure complete dissolution of the drug. This mixture was added dropwise into 133 mL of an aqueous solution of PLGA-PEG-PLGA triblock copolymer (Example 3; 20%w/w). The solution was sterile-filtered through a 0.2 μ filter before being dispensed into vials so that each vial contained 6 g of the solution. Water in the solution was removed by lyophilization. The residue was homogenized by rotating on a roller at 50° C. for 2 hours. After temperature equilibrated to ambient, water for injection (4.6 g) was added to the mixture. The mixture was subjected to wrist shaking at approximately 100 strokes/min. The solution was visually inspected every 30 seconds for homogeneity. The mixture took 1 minute to reconstitute. The gelation temperature of the solution was 29.2° C.

### COMPARATIVE EXAMPLE OF EXAMPLE 14

Paclitaxel (1.04 g) was added to an aqueous solution of PLGA-PEG-PLGA triblock copolymer synthesized in Example 3 (133 mL, 20%). The mixture was allowed to stir at ambient temperature for ca. 3 hours. It was then sterile-filtered through a 0.2 μ filter and dispensed into vials. Each vial received 4.6 g of the solution. Water in the solution was removed by lyophilization. Reconstitution of the residue was examined by addition of 4.6 g of water for injection. Wrist shaking at approximately 100 stroke/min did not dissolve the content in 15 minutes. The mixture was further mixed using a magnetic stirring at ca. 200 rpm at ambient conditions. Complete dissolution took longer than 5 hours. The gelation temperature was 29.8° C.

### EXAMPLE 15

PEG-300 (40 g) was placed in a 250-mL round bottom flask. Moisture was removed by drying under vacuum (0.2 torr) at 90° C. for 3 hours. Acetic anhydride (30 g) was added and the reaction mixture was brought to reflux under nitrogen over 48 hours. Excess acetic anhydride was removed by vacuum distillation at 100° C. for 24 hours. The reconstitution-enhancing and enabling agent was obtained by blending the residue with PEG 300 at 3:1 ratio.

Polymer synthesized by the method described in Example 3 (1 g) was intimately mixed with the reconstitution-enhancing agent (1.5 g). Water for injection (5 g) was added to the mixture. Reconstitution of the polymer mixture took less than 1 minute. The gelation temperature of the solution was 31.8° C.

### EXAMPLE 16

Polymer synthesized in Example 2 (1 g) was added 0.5 g of PEG 200, 1 g of tri(ethylene glycol) monomethyl ether and 10 mg of paclitaxel. The mixture was homogenized by mixing at ca. 45° C. After cooled to ambient, the mixture was added 5 mL of water, reconstitution of the mixture took less than 1 minute. The solution was clear, free-flowing liquid at room temperature and body temperature.

### EXAMPLE 17

Testosterone enanthate (500 mg) was suspended in 8 ml of water. Five mL of the suspension were added to a homogeneous mixture comprised of 1 gram of triblock copolymer prepared in Example 3 and 1.5 gram of reconstitution enhancing and enabling agent prepared in Example 1. Reconstitution of the mixture took 1 minute. The resulting suspension had a gelation temperature at 31.4° C.

The above description will enable one skilled in the art to make a composition comprising biodegradable block copolymer drug carriers and a reconstitution enhancing and enabling agent comprising a low molecular weight PEG, a PEG derivative, or a mixtures of PEG and PEG derivative, said composition can be rapidly reconstituted in an aqueous vehicle to afford useful forms that may be either homogeneous solutions or uniform colloidal systems. Although the reconstitutable compositions are illustrated in the examples to show the functionality of the compositions of the present invention, these descriptions are not intended to be an exhaustive statement of all drug carriers that can be rendered constitutable by addition of the reconstitution agents of the present invention. Certainly, numerous other drug carriers or drugs from various categories of therapeutic agents are well suited for the reconstitutable drug delivery compositions described in this invention. It will be immediately apparent to one skilled in the art which various modifications may be made without departing from the scope of the invention that is limited only by the following claims and their functional equivalents.

We claim:

1. A composition having enhanced reconstitution properties comprising:
1) one or more biodegradable block copolymer drug carriers comprising A-B, A-B-A or B-A-B block copolymers having a total weight average molecular weight of 2000 to 8000 Daltons, wherein the A block is a biodegradable polyester or poly(ortho ester) and the B block is polyethylene glycol(PEG), and the weight percentage of the A block is between 50.1% to 83% and the weight percentage of the B block is between 17% to 49.9%; and
2) a reconstitution enhancing and enabling agent comprising polyethylene glycol(PEG), a PEG derivative, or a mixtures of PEG and a PEG derivative, said PEG or PEG derivative having a molecular weight of 150 to 1100 Daltons;
wherein the biodegradable block copolymeric drug carrier is soluble in an aqueous solution and miscible with the reconstitution enhancing and enabling agent.

2. The composition according to claim 1 wherein the PEG derivative is comprised of PEG that has been derivatized with a member selected from the group consisting of D,L-lactide, D-lactide, L-lactide, D,L-lactic acid, D-lactic acid, L-lactic acid, glycolide, glycolic acid and copolymers thereof.

3. The composition according to claim 1 wherein the PEG derivative is represented by R¹—CO—O-(PEG)-CO—R² or R¹—O-(PEG)-R² wherein R¹ and R² are independently members selected from the group consisting of H and C₁ to C₁₀ alkyl.

4. The composition according to claim 1 wherein the biodegradable block copolymeric drug carrier further comprises a biodegradable polyester or poly(ortho ester) oligomer.

5. The composition according to claim 1 wherein the weight ratio of the biodegradable block copolymeric drug carrier and the reconstitution enhancing and enabling agent is within the range of 2:1 to 1:99.

6. The composition according to claim 1 further comprising a drug.

7. The composition according to claim 1, wherein said composition can be reconstituted in water or an aqueous solution to form a homogeneous solution or an uniform colloidal system within 0.01 minutes to 180 minutes.

8. A composition having enhanced reconstitution properties comprising:
1) one or more biodegradable ABA- or BAB-type tri-block copolymers comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol(PEG), and wherein the tri-block copolymer having a weight average molecular weight of between about 2000 to 4990 and possessing reverse thermal gelation properties, and
2) a reconstitution enhancing and enabling agent comprising a polyethylene glycol(PEG), a PEG derivative, or a mixture of PEG and a PEG derivative, said PEG or PEG derivative having a weight averaged molecular weight of 150 to 1100 Daltons, and
wherein the biodegradable block copolymeric drug carrier is soluble in an aqueous solution and miscible with the reconstitution enhancing and enabling agent.

9. The composition according to claim 8 wherein the PEG derivative is comprised of PEG that has been derivatized with a member selected from the group consisting of D,L-lactide, D-lactide, L-lactide, D,L-lactic acid, D-lactic acid, L-lactic acid, glycolide, glycolic acid and copolymers thereof.

10. The composition according to claim 8 wherein the PEG derivative is represented by R¹—CO—O-(PEG)-CO—R² or R¹—O-(PEG)-R² wherein R¹ and R² are independently members selected from the group consisting of H and C₁ to C₁₀ alkyl.

11. The composition according to claim 8 wherein the biodegradable block copolymeric drug carriers further comprise a biodegradable polyester or poly(ortho ester) oligomer.

12. The composition according to claim 8 wherein the weight ratio of the biodegradable block polymeric drug carrier and the reconstitution enhancing and enabling agent is within the range of 2:1 to 1:99.

13. The composition according to claim 8 further comprising a drug.

14. The composition according to claim 8, wherein said composition can be reconstituted in water or an aqueous solution to form a homogeneous solution or an uniform colloidal system within 0.01 minutes to 180 minutes

15. A composition having enhanced reconstitution properties comprising:
1) a biodegradable ABA-type, BAB- or AB-type block copolymer, comprising:
i) 50.1 to 65% by weight of a biodegradable, hydrophobic A block comprising a biodegradable polyester or poly(ortho ester), and
ii) 35 to 49.9% by weight of a hydrophilic B block comprising a polyethylene glycol (PEG), and wherein the block copolymer has a weight average molecular weight of between 2400 to 4999 daltons, with the proviso that said block copolymer, when formed as an aqueous polymer solution, is a free flowing liquid at temperatures of between at least 35 to 42° C., and
2) a reconstitution enhancing and enabling agent comprising polyethylene glycol(PEG), a PEG derivative, or a mixture of PEG and a PEG derivative, said PEG or PEG derivative having a weight averaged molecular weight of 150 to 1100 Daltons, and
wherein the biodegradable block copolymeric drug carrier is soluble in an aqueous solution and miscible with the reconstitution enhancing and enabling agent.

16. The composition according to claim 15 wherein the PEG derivative is comprised of PEG that has been derivatized with a member selected from the group consisting of D,L-lactide, D-lactide, L-lactide, D,L-lactic acid, D-lactic acid, L-lactic acid, glycolide, glycolic acid and copolymers thereof.

17. The composition according to claim 15 wherein the PEG derivative is represented by R¹—CO—O-(PEG)-CO—R² or R¹—O-(PEG)-R² wherein R¹ and R² are independently members selected from the group consisting of H and C₁ to C₁₀ alkyl.

18. The composition according to claim 15 wherein the biodegradable block copolymeric drug carrier further comprises a biodegradable polyester or poly(ortho ester) oligomer.

19. The composition according to claim 15 wherein the weight ratio of the biodegradable block copolymeric drug carrier and the reconstitution enhancing and enabling agent is within the range of 2:1 to 1:99.

20. The composition according to claim 15 further comprising a drug.

21. The composition according to claim 15, wherein said composition can be reconstituted in water or an aqueous solution to form a homogeneous solution or an uniform colloidal system within 0.01 minutes to 180 minutes

22. A composition having enhanced reconstitution properties comprising:
1) a biodegradable polyester or poly(ortho ester) oligomer having a weight average molecular weight of between 400 and 10,000 daltons; and a biodegradable AB-type, ABA-type, or BAB-type block copolymer comprising:
i) 50.1 to 65% by weight of a biodegradable, hydrophobic A block comprising a biodegradable polyester or poly(ortho ester), and
ii) ii) 35 to 49.9% by weight of a hydrophilic B block comprising a polyethylene glycol (PEG), and wherein the block copolymer has a weight average molecular weight of between 2400 to 4999 daltons, and
2) a reconstitution enhancing and enabling agent comprising polyethylene glycol(PEG), a PEG derivative or a mixture of PEG and a PEG derivative, said PEG or PEG derivative having a weight averaged molecular weight of 150 to 1100 Daltons, and
wherein the biodegradable block copolymeric drug carrier is soluble in an aqueous solution and miscible with the reconstitution enhancing and enabling agent.

23. The composition according to claim 22 wherein the PEG derivative is comprised of PEG that has been derivatized with a member selected from the group consisting of D,L-lactide, D-lactide, L-lactide, D,L-lactic acid, D-lactic acid, L-lactic acid, glycolide, glycolic acid and copolymers thereof.

24. The composition according to claim 22 wherein the PEG derivative is represented by R¹—CO—O-(PEG)-CO—R² or R¹—O-(PEG)-R² wherein R¹ and R² are independently members selected from the group consisting of H and C₁ to C₁₀ alkyl.

25. The composition according to claim 22 wherein the biodegradable block copolymeric drug carrier further comprises a biodegradable polyester or poly(ortho ester) oligomer.

26. The composition according to claim 22 wherein the weight ratio of the biodegradable block copolymeric drug carrier and the reconstitution enhancing and enabling agent is within the range of 2:1 to 1:99.

27. The composition according to claim 22 further comprising a drug.

28. The composition according to claim 22, wherein said composition can be reconstituted in water or an aqueous solution to form a homogeneous solution or an uniform colloidal system within 0.01 minutes to 180 minutes.

29. A method of reconstituting a drug delivery composition comprising the steps of:
A) providing a composition having enhanced reconstitution properties comprising:
1) one or more biodegradable polymeric block copolymer drug carriers comprising A-B, A-B-A or B-A-B block copolymers having a total weight average molecular weight of 2000 to 8000 Daltons, wherein the A block is a biodegradable polyester or poly(ortho ester) and the B block is polyethylene glycol(PEG), and the weight percentage of the A block is between 50.1 to 83 and the weight percentage of the B block is between 17% to 49.9%; and
2) a reconstitution enhancing and enabling agent comprising a polyethylene glycol(PEG), a PEG derivative or a mixtures of PEG and a PEG derivative, said PEG or PEG derivative having a weight averaged molecular weight of 150 to 1100 Daltons;
wherein the biodegradable block copolymeric drug carrier is soluble in an aqueous solution and miscible with the liquid reconstitution enhancing and enabling agent; and
B) mixing water or an aqueous solution with the composition to form a homogeneous solution or an uniform colloidal system.

30. The method according to claim 29 wherein the PEG derivative is comprised of PEG that has been derivatized with a member selected from the group consisting of D,L-lactide, D-lactide, L-lactide, D,L-lactic acid, D-lactic acid, L-lactic acid, glycolide, glycolic acid and copolymers thereof.

31. The method according to claim 29 wherein the PEG derivative is represented by R¹—CO—O-(PEG)-CO—R² or R¹—O-(PEG)-R² wherein R¹ and R² are independently members selected from the group consisting of H and C₁ to C₁₀ alkyl.

32. The method according to claim 29 wherein the biodegradable drug block copolymeric carrier further comprises a biodegradable polyester or poly(ortho ester) oligomer.

33. The method according to claim 29 wherein the weight ratio of the biodegradable block copolymeric drug carrier and the reconstitution enhancing and enabling agent is within the range of 2:1 to 1:99.

34. The method according to claim 29, wherein the composition further comprising a drug.

35. The method according to claim 29 wherein the ratio of the composition to water or aqueous solution is within the range of 2:1 to 1:1000.

36. The method according to claim 29 wherein the composition can be reconstituted in water or an aqueous solution to form a homogeneous solution or an uniform colloidal system within 0.01 minutes to 180 minutes.
